(19) United States
(12) Patent Application Publication
(10) Pub. No.: US 2025/0065335 A1
(43) Pub. Date: Feb. 27, 2025

(54) LID FOR ASSAY AND MICROTITER PLATES

(71) Applicant: MESO SCALE TECHNOLOGIES, LLC., Rockville, MD (US)

(72) Inventors: Manish KOCHAR, Rockville, MD (US); Gary KRIVOY, Rockville, MD (US); Robert CAUSEY, Rockville, MD (US); Slavi MIRTCHEV, Gaithersburg, MD (US)

(21) Appl. No.: 18/667,266

(22) Filed: May 17, 2024

Related U.S. Application Data
(60) Provisional application No. 63/502,991, filed on May 18, 2023.

(51) Int. Cl. B01L 3/00

(52) U.S. Cl. CPC B01L 3/50853; B01L 2200/025; B01L 2300/0829; B01L 2300/0858

(57) ABSTRACT

Devices and methods for a microplate lid configured to cover a microwell assay plate are provided. A microplate lid includes a lid panel surrounded by a lid frame. Sidewalls extend downwardly from lid frame. The microplate lid includes various features to improve performance, including features to improve stickability, reduce warping, reduce sample contamination, reduce wear, reduce sample evaporation, facilitate robotic handling, and provide other benefits.

### CROSS-REFERENCE TO RELATED APPLICATION

The present patent application claims priority to U.S. Provisional Patent Application entitled “LID FOR ASSAY AND MICROTITER PLATES” bearing application No. 63/502,991 and filed on May 18, 2023, the entire contents of which are incorporated herein by reference.

### FIELD OF THE INVENTION

The present invention is directed to a microwell assembly, and more specifically to a microplate lid designed for maximizing reliability and functionality.

### BACKGROUND

Some microwell assemblies include a microplate lid and a microwell assay plate, wherein the microwell assay plate is sterilized and includes multiple reservoirs for holding specimens therein. Microplate lids are designed to fit over these the microwell assay plates, to shield the reservoirs or wells (and held specimens) from outside elements to avoid compromise/contamination.

Microplate lids may frequently exhibit characteristics undesirable for sample processing, including manufacturing inconsistencies, unintended curvatures (e.g., warping), excessive wear, and others.

It is thus desirable to provide a microplate lid that may be manufactured (e.g. molded, formed, constructed, 3D printed, extruded, etc.) to maintain a desirable shape, be resistant to warping, reduce excess wear, and provide further features that facilitate sample processing.

### SUMMARY

Embodiments consistent with the present disclosure provide a microplate lid for microwell assay plates adapted and configured to address problems described herein. Microplate lids consistent with the present disclosure may feature a greater resistance to wear, may include improved stacking and handling features, may include increased protection against sample compromise, may include features that facilitate robotic and manual handling, and may include features that improve manufacturing consistency. Microplate lids consistent with the present disclosure may include any combination of these features and elements.

In an embodiment, a microplate lid configured to cover a microwell assay plate is provided. A lid panel having a top panel surface and a bottom panel surface, a lid frame surrounding the lid panel having a top frame surface and a bottom frame surface; and a plurality of sidewalls extending from a periphery of the bottom frame surface; and further comprising at least one feature selected from: a plurality of ribs extending from the top frame surface, chamfered edges disposed at a bottom of the plurality of sidewalls, a convex shape formed by the lid panel, and a substantially flat contact surface formed by the frame bottom surface.

### BRIEF DESCRIPTION OF THE DRAWINGS

The accompanying figures, which are incorporated herein, form part of the specification and illustrate embodiments of microplate lids for microwell assay plates. Together with the description, the figures further explain the principles of and enable a person skilled in the relevant art(s) to make and use the devices described herein. The drawings are provided to illustrate various features of the embodiments described herein and are not necessarily drawn to scale. In the drawings, like reference numbers indicate identical or functionally similar elements.

FIG. 1 is a top-down perspective view of a microwell assay plate lid.

FIGS. 2A and 2B illustrate a microplate lid consistent with embodiments hereof.

FIGS. 3A and 3B illustrate features of a microplate lid panel consistent with embodiments hereof.

FIG. 3C is a front elevational cross-sectional view of a microwell assay plate and lid.

FIG. 3D is a close up, front elevational cross-sectional view of the microwell assay play and lid of feature A depicted in FIG. 3C.

FIG. 3E illustrates two microplate lids in a stacked configuration, consistent with embodiments hereof.

FIG. 4 illustrates features of a microplate lid frame consistent with embodiments hereof.

FIGS. 5A-5C illustrate features of a microplate lid frame consistent with embodiments hereof.

FIG. 6 illustrates features of a microplate lid consistent with embodiments hereof.

FIG. 7 illustrates features of an automated assay system consistent with embodiments hereof.

### DETAILED DESCRIPTION

The following detailed description is merely illustrative in nature and is not intended to limit the invention or the application and uses of the invention. Although the description of the invention is in the context of devices, such as microplate lids, for use with microwell assay plates, the disclosure should not be considered so limiting. For example, the lids described herein may be suitable or useful for covering, protecting, etc., any type of substrate, plate, container, etc. Modifications may be made to the embodiments described herein without departing from the spirit and scope of the present invention. Therefore, the following detailed description is not meant to be limiting. Furthermore, there is no intention to be bound by any expressed or implied theory presented in the preceding technical field, background, summary, or the following detailed description.

The present disclosure may include references to relative terms such as “top,” “bottom,” “up,” and “down.” These terms are used for clarity and ease of reference. For example, a “top” of a structure or device may refer to the portion of that structure or device that faces up during usage as described herein. Relative directional terms as used herein are not limiting and do not limit the orientations, positions, angles, or functionality of the structures and devices discussed herein. Furthermore, the methods, systems, and devices discussed herein are not limited to use in the orientations as described herein. For example, although the microplate lid is described as having a top surface and a bottom surface, the disclosure is not limited to this arrangement. One or more aspects of the devices described herein may be inverted with respect to the orientations disclosed herein without departing from the scope of this disclosure.

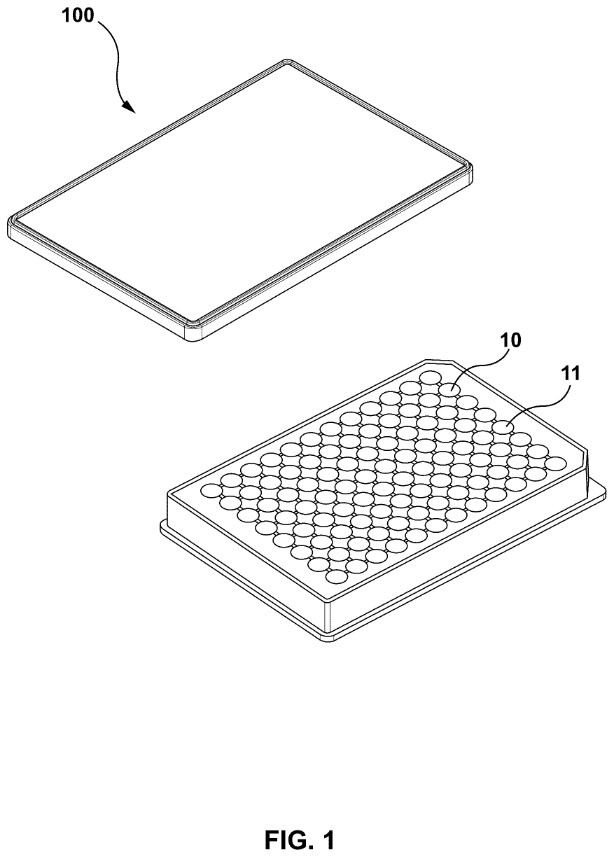

FIG. 1 illustrates a microplate lid configured for use with a microwell assay plate. The microplate lid 100 is configured to rest atop the microwell assay plate 10 and to cover the wells 11 located therein. As described in further detail below, features of various embodiments as described herein may address specific issues that have been identified with the use of a conventional or standard microplate lid 100 with the microwell assay plate 10.

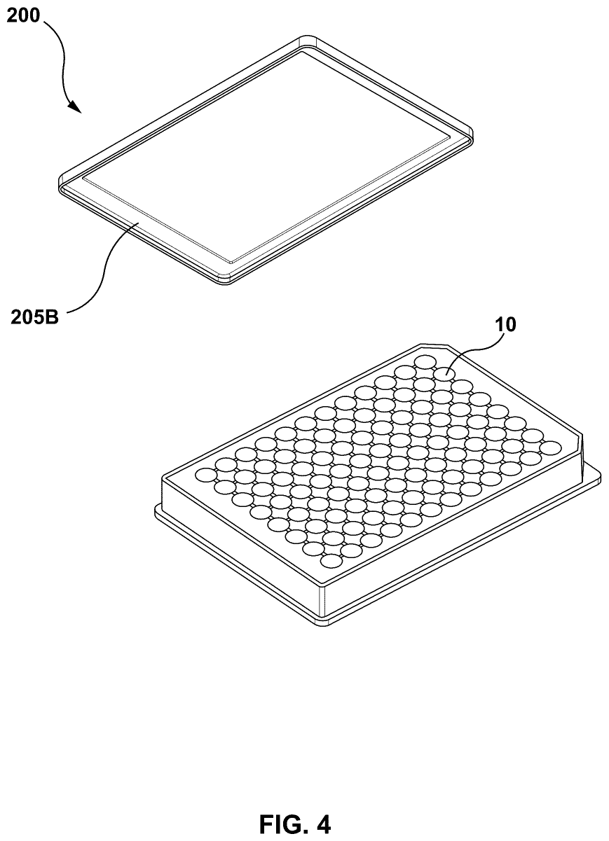

The microwell assay plate 10 may include any number of wells 11. For example, as illustrated in FIG. 1 and below in FIG. 4, the microwell assay plate 10 may include 96 wells 11. One skilled in the art will realize that the microwell assay plate 10 may include any number of wells 11 (e.g. 6 wells, 24, 384, 1536, etc.) manufactured in a regular or irregular pattern. In other embodiments, the microwell assay plate 10 may be replaced by a single-well plate or any other apparatus suitable for conducting biological, chemical, and/or biochemical analysis and/or assays. Although wells 11 are depicted in FIGS. 1 and 4 in a circular configuration (thus forming cylinders) other shapes are contemplated as well, including ovals, squares, and/or other regular or irregular polygons. Further, the shape and configuration of microwell assay plate 10 may take multiple forms and is not necessarily limited to a rectangular array as illustrated in these figures.

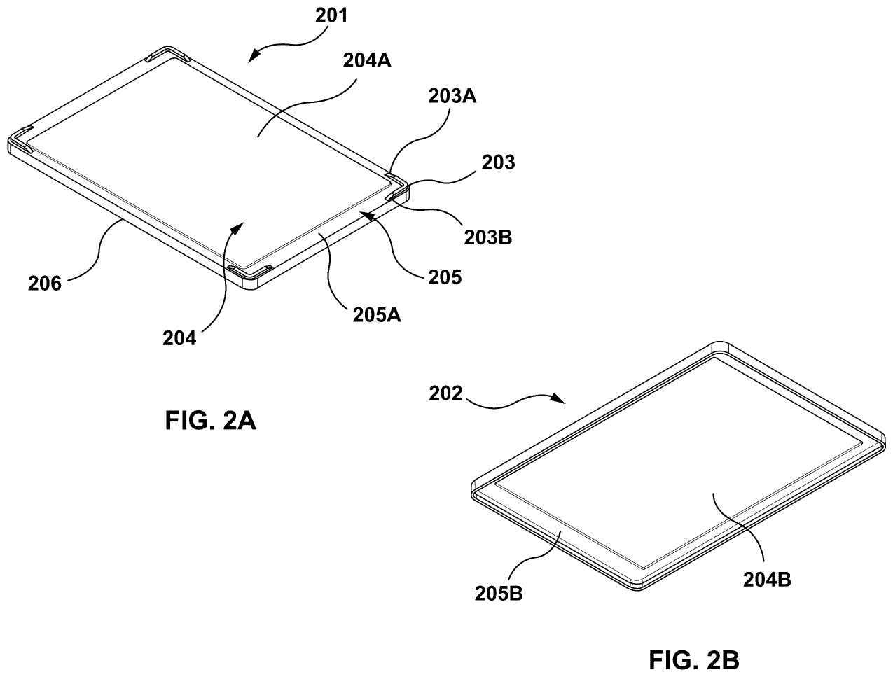

FIGS. 2A and 2B illustrate a microplate lid 200 consistent with embodiments hereof. In embodiments, the microplate lid 200 may be provided for use with one or more microwell assay plates 10 (as shown in FIG. 1). In embodiments, the microplate lid 200 and a microwell assay plate 10 may be provided together as an assay system.

In embodiments, the microplate lid 200 may be configured to cover the microwell assay plate 10. It will be understood that the below-described features of the microplate lid 200 may be altered in size and shape to accommodate microwell assay plates having alternative form factors (more or fewer wells, different sizes, different shapes, etc.) without departing from the scope of this disclosure.

As shown in FIGS. 2A and 2B, the microplate lid 200 includes lid panel 204 and a lid frame 205 surrounding the lid panel 204. The lid panel 204 includes a top panel surface 204A and bottom panel surface 204B. The lid frame 205 includes a top frame surface 205A and a bottom frame surface 205B. The top panel surface 204A and the top frame surface 205A define a top surface 201 of the microplate lid 200. The bottom panel surface 204B and the bottom frame surface 205B define a bottom surface 202 of the microplate lid 200. A plurality of sidewalls 206 extend downwards from the lid frame 205 in an approximately perpendicular orientation (for example, between 90-94 degrees), forming a substantially continuous circumference about the microplate lid 200. The approximately perpendicular orientation of the sidewalls 206 is selected to ensure that the plurality of sidewalls 206 form an angle that is 90 degrees or larger. Angles slightly larger than perpendicular can assist when placing the lid atop a microwell assay plate 10.

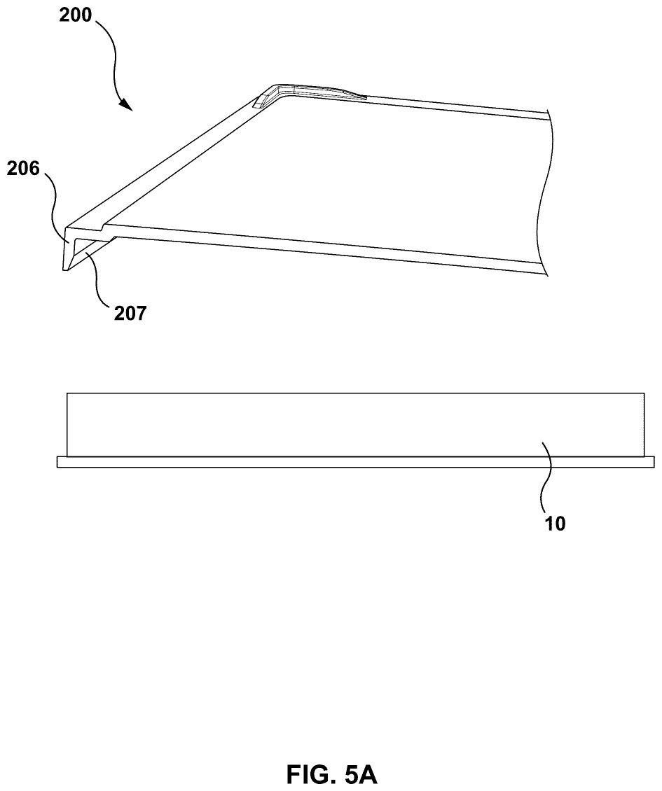

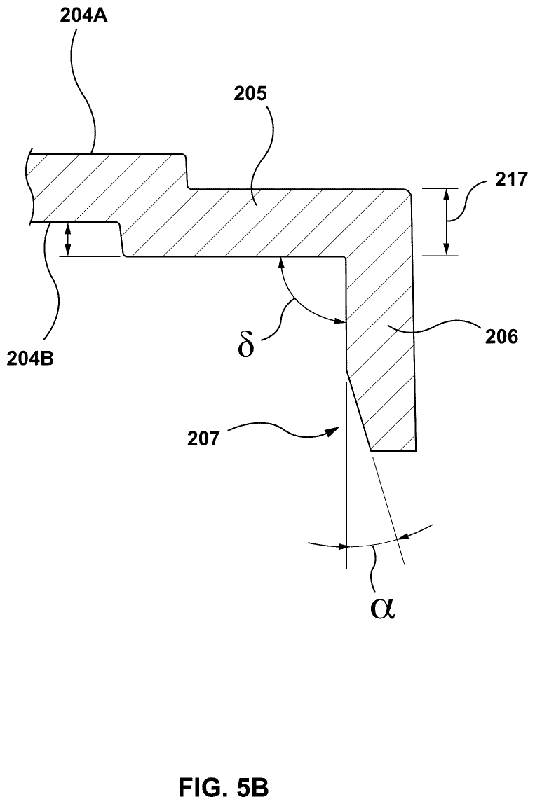

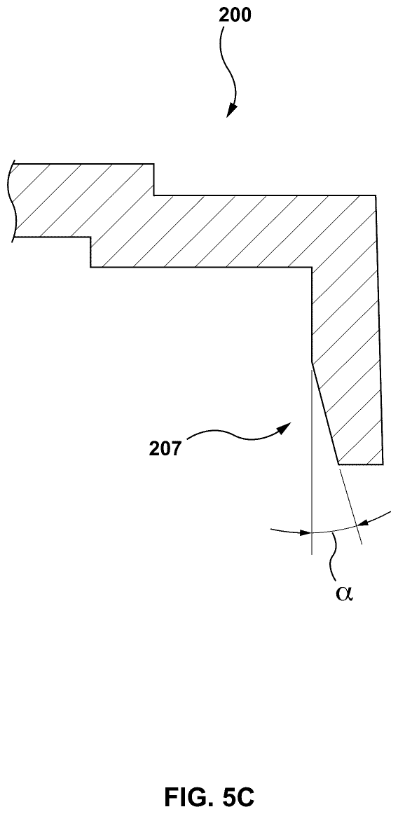

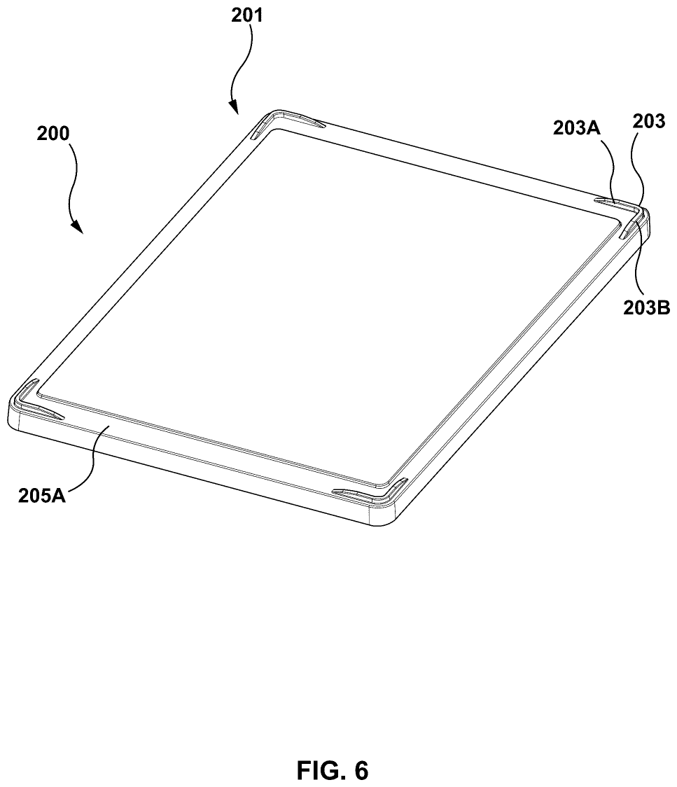

The top surface 201 includes a plurality of ribs 203 which extend upwards from the top frame surface 205A, wherein each rib 203 may include perpendicular extensions 203A, 203B protruding therefrom. The ribs 203 are described in greater detail with respect to FIG. 6. The sidewalls 206 include chamfered edge 207, which are described in greater detail with respect to FIGS. 5A-6. The microplate lid 200 is sized to receive—or cover the top surface of—a microwell assay plate 10, the microwell assay plate 10 which includes wells 11 for receiving samples therein. As will be described in greater detail below, the microplate lid 200 presents multiple advantages over conventional or standard microplate lids 100, including, but not limited to: resistance to warping, resistance to wear, case of robotic placement, improved stacking and handling features, and increased protection against specimen compromise.

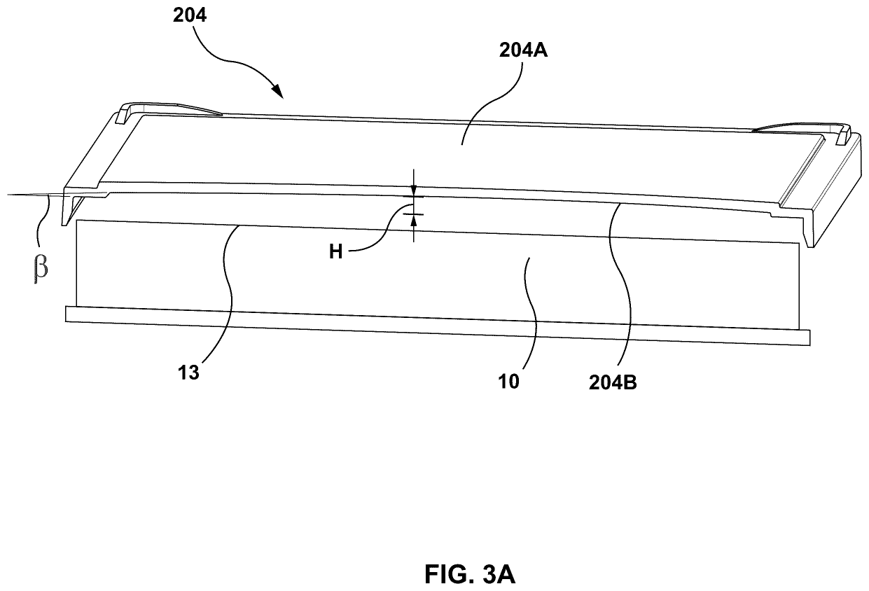

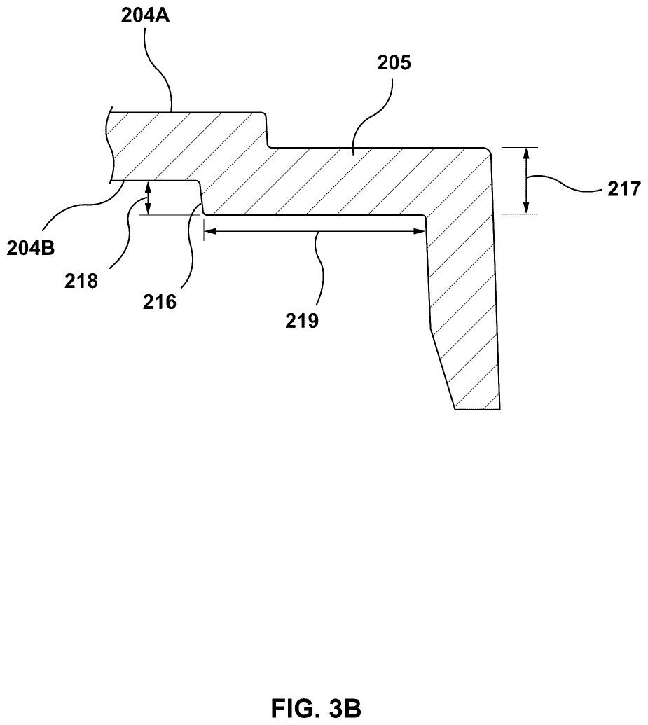

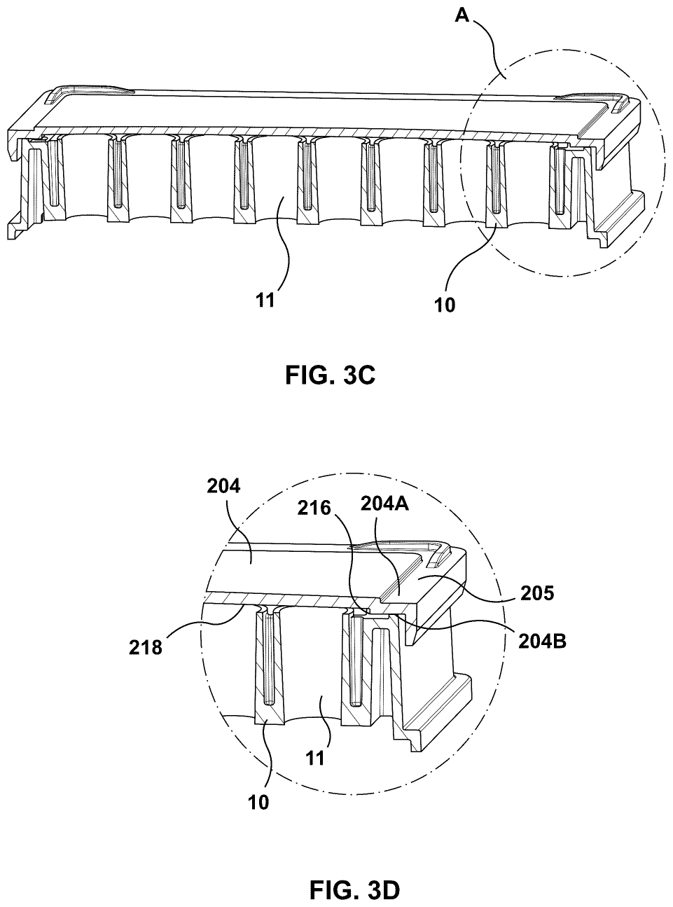

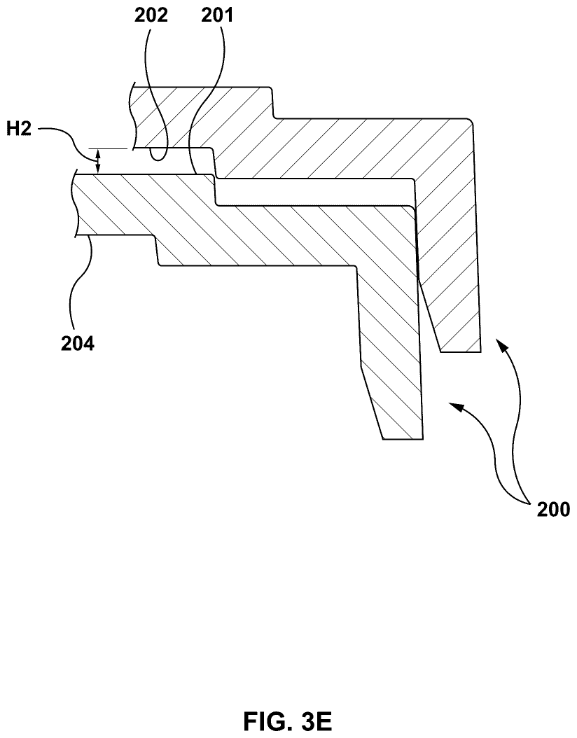

FIG. 3A illustrates features of the lid panel 204 of the microplate lid 200 consistent with embodiments hereof. FIG. 3B illustrates a profile of the microplate lid 200 edge. In particular, FIG. 3A highlights aspects of the shape of the microplate lid 200. The lid panel 204 of the microplate lid 200 may be disposed adjacent to and vertically offset from the lid frame 205, such that it is surrounded by the lid frame 205. The lid panel 204 includes a raised or convex dome shape, having a spacing height H. As used herein, the term “dome shape” refers to the lid panel 204 being raised in a central portion. In some embodiments, the dome shape may be formed by a curvature in the lid, as discussed below. In some embodiments, the dome shape may be formed from flat sections of the lid panel 204 that extend from the lid frame 205 at a non-zero angle and meet together at a non-zero angle. In some embodiments, the dome shape may be formed from a combination of flat and curved portions to achieve the spacing height H. The lid panel 204 may include a raised or convex dome shape in either a longitudinal dimension, a lateral dimension, or both. The convex or dome like shape of the lid panel 204 causes it to have a spacing height H along a longitudinal center line, a lateral center line, or a center of the lid panel 204, depending on whether the lid panel 204 is domed in a longitudinal dimension, a lateral dimension, or both. The spacing height H represents a distance between the bottom panel surface 204B of the lid panel 204 and the plane defined by the outer circumference of the bottom panel surface 204B of the lid panel 204. The spacing height H represents a distance between the height of the bottom panel surface 204B of the dome shaped lid panel 204 and the height of the bottom surface of a flat lid panel (e.g., a lid panel that is flat and undomed). The spacing height H may be approximately 0.02 in (or between 0.01 and 0.03 in). The top panel surface 204A of the lid panel 204 may have a spacing height similar to the spacing height H of the bottom panel surface 204B, comparatively represented by the distance between the height of the top panel surface 204A of the dome shaped lid panel 204 and the height of the top surface of a flat lid panel. In embodiments, the top panel surface 204A of the lid panel 204 may have a spacing height dissimilar to the spacing height H of the bottom panel surface 204B, the dissimilar spacing height represented by the distance between the height of the top panel surface 204A of the dome shaped lid panel 204 and the height of the top surface of a flat lid panel. This dissimilar height may be the result of more/less material used in manufacturing certain portions of the lid panel 204, resulting in a variable thickness throughout the lid panel 204.

As noted above, the lid panel 204 may further be offset vertically from the lid frame 205 by an offset dimension 218, which may be approximately 0.02 in (or between 0.01 and 0.03 in.). The offset dimension 218 may prevent condensate or splashing liquid from the wells 11 accumulating or building up on the lid panel 204 from contacting the microwell assay plate 10, and further prevents such liquid buildup from dripping into other wells 11, thus preventing cross contamination. Liquid accumulating on the lid panel 204 will instead traverse towards the lid frame 205 and away from the wells 11. The offset dimension 218 further prevents this liquid run off from dripping into the wells 11 located near the perimeter of the microwell assay plate 10, as liquid gathered near the edges of the bottom panel will then traverse down the angle formed by corners 216, and drip into an area past the wells 11 (as shown in FIGS. 3B-3D). Further the lid frame may have a thickness dimension 217 of approximately 0.04 in. (or between 0.03 and 0.05 in.). Together, these dimensions may stack up to raise the highest point of the bottom panel surface 204B of the panel 204 to have a total distance of approximately 0.05 in (or between 0.03 and 0.07 in.) above the bottom frame surface 205B. Accordingly, when the microplate lid 200 rests on a microwell assay plate 10, supported by the interface between the bottom frame surface 205B and a top surface 13 of the microwell assay plate 10, a total maximum distance created between the bottom panel surface 204B of the lid panel 204 and the top surface 13 of the assay plate is approximately 0.05 in (or between 0.03 and 0.07 in.). This maximum distance is achieved along a latitudinal or longitudinal center line of the microplate lid 200.

The dome may be achieved, for example, by constructing the lid panel 204 such that it projects from the top frame 205 according to a curvature. Thus, the dome of the lid panel 204 may be achieved by the lid panel 204 conforming to any appropriate curve. The lid panel 204 may have a radius of curvature. The radius of curvature may be constant and/or may vary according to a position along the curve of the dome of the lid panel 204. Accordingly, it is not required that the dome have a circular profile or a be a portion of a sphere, although it may be. Further, the lid panel 204 may have an elliptical curvature or any other suitable curvature that achieves the appropriate height H. The lid panel 204 may have a curvature in a longitudinal dimension, a lateral dimension, or both. The curvature may be a constant curvature or may be a variable curvature.

In embodiments, the lid panel 204 may form an angle β that is non-zero in relation to a plane defined by at least one of the inner perimeter of the lid frame 205 or the outer perimeter of the lid panel 204 (e.g., where it overlaps with the lid frame 205). In embodiments, the angle β may be formed on all four sides of the lid panel 204. In embodiments, the angles formed on the four sides of the lid panel 204 may be non-equal with respect to each other, such that the angle β of a first two sides of the all four sides is greater than the angle β of opposing two sides, creating a more pronounced taper/dome of the lid panel 204 on the side of the microplate lid 200 adjacent to the first two sides, as opposed to the portion of the microplate lid 200 adjacent to the opposing two sides. In embodiments, the lid panel 204 may be domed according to a combination of an angle β at the perimeter of the lid panel 204 and a radius of curvature of the lid panel 204. In embodiments, the lid panel 204 may be formed from a several flat sections. For example, four flat triangular sections may form a dome with a pyramid shape. Such a pyramid shape may include a flattened top (e.g., a fifth square or rectangular section that meets the triangular sections). In another example, two flat trapezoidal and two flat triangular sections may form a dome.

A flat assay lid (e.g., an assay lid having a zero β angle and no dome shape) may have a tendency to warp or bend, and may warp or bend inward. In such a situation, the bottom surface of the flat assay lid may bend inward enough to contact samples within the wells 11 of a microwell assay plate 10. Such contact may cause cross-contamination of samples via fluid bridging between wells 11 and/or may cause the lid to stick to the microwell assay plate 10. The domed shape of the lid panel 204 having the spacing height H may provide at least three advantages over such flat assay lids. First, the domed shape of the lid panel 204 provides warping resistance—particularly against inward/concave warping scenarios that may otherwise result in compromise of the samples on or within the assay plate. Second, the domed shape of the lid panel 204 may act as a more stable profile and more effectively prevents subsiding when compared to flat assay lids. Third, the spacing height H facilitates the prevention of contact between samples contained within the microwell assay plate 10 and the bottom panel surface 204B. Fourth, the domed shape of the lid panel 204 provides a path for any liquid that does contact the bottom panel surface 204B (e.g., via evaporation/condensation and/or splashing) to run off to the edges of the bottom panel surface 204B and avoid sample contact. The offset dimension 218 further prevents this liquid run off from dripping into the wells 11 located near the perimeter of the microwell assay plate 10, as liquid gathered near the edges of the bottom panel will then traverse down the angle formed by corners 216, and drip into an area past the wells 11 (as shown in FIGS. 3B-3D). Furthermore, the domed shape of the lid panel 204 prevents liquid bridging between the lid panel 204 and the microwell assay plate 10. Liquid bridging, especially from evaporation/condensation, can cause the microwell assay plate 10 to be raised along with the lid panel 204 during manual or automated/robotic handling as if they are adhered to each other. When the liquid bridge is broken during such handling, the microwell assay plates 10 may then detach from the lid pane 204 and inadvertently drop from a height. Such handling mistakes waste precious patient or other samples, or can cause a crash or failure in an automated system. Thus, avoiding or reducing liquid bridging reduces numerous handling mistakes.

A flat assay lid (i.e. where angle β is zero and there is no dome shape) may have a tendency to warp or bend, and may warp or bend inward. In such a situation, the bottom surface of the flat assay lid may bend inward enough to contact samples within the wells 11 of a microwell assay plate 10. Such contact may cause cross-contamination of samples via fluid bridging between wells 11 and/or may cause the lid to stick to the microwell assay plate 10.

FIG. 3E illustrates how the domed shape of the lid panel 204 facilitates stacking of multiple microplate lids 200 by providing a spacing height H2 between the top surface 201 and the bottom surface 202 of another lid 200 stacked on top of the first lid, such that the dome does not contact the stacked lid bottom panel surface 204. The stacking capabilities of the microplate lid 200 of the present invention will be described in greater detail below.

FIG. 4 illustrates a bottom-up perspective view of a microwell assay plate 10 and the microplate lid 200 consistent with embodiments hereof. The bottom frame surface 205B is manufactured (e.g. molded, formed, constructed, 3D printed, extruded, etc.) with a substantially flat, substantially planar surface and provides a substantially continuous contact surface in substantially continuous contact (e.g., continuous contact with less than 10%, less than 5%, less than 2%, less than 1%, etc. interruptions as measured by perimeter percentage.) with the perimeter of the microwell assay plate 10 upon disposal of the microplate lid 200 over the top of the microwell assay plate 10. This may result in the substantially all of the bottom frame surface 205B being in contact with the microwell assay plate 10. Substantially all may include more than 90%, more than 95%, more than 98%, or more than 99% of the bottom frame surface 205B as measured by area. By substantially planar, it is meant that bottom frame surface 205B is not more than 0.013 in. out of plane. By substantially flat, it is meant that that the bottom frame surface 205B has a surface flatness of less than 0.013 in. The bottom frame surface 205B may be approximately 0.275 in (or between 0.25 in and 0.30 in) in a width dimension 219 (see FIG. 3B) around the perimeter. In embodiments, the bottom frame surface 205B may have different widths along the longitudinal sides and on the lateral sides.

The features of the lid frame 205 of the microplate lid may address issues present in alternative microplate lids (described in greater detail below) in several ways. A few non-limiting examples of those benefits are provided below. As a first example, relative to other microplate lid designs, the microplate lid 200 has significantly increased contact area with the surface of the microwell assay plate 10, due to the width of the bottom frame surface 205B and the substantially continuous nature of the contact, as described above. This increase in contact area causes the gripping interaction between the microplate lid 200 and the microwell assay plate 10 to be substantially increased, which may reduce the amount of lid wear during shaking operations. During experimental shaking operations, powder buildup was found to be substantially decreased or eliminated entirely with respect to other microplate lid designs through use of the microplate lid 200 featuring the bottom frame surface 205B.

In contrast, in some conventional microplate lids, raised ribs along the periphery on the underside of the lid are used as contact surfaces with a microwell assay plate 10. In such microplate lids, the ribs are configured to achieve a gap between the underside of the microplate lid and the top of the microwell assay plate. During shaking sequences, these ribs may rub against the microwell assay plate and wear. This wear may create a fine powder that can contaminate samples or machinery and may reduce the gap between the underside of the microplate lid and the top of the microwell assay plate, thus exacerbating the above-discussed issue with fluid bridging. In an additional difference, in some conventional microplate lids, these ribs may not be continuous, which may serve to increase air flow through the volume between the microplate lid and the microwell assay plate 10. Such airflow can cause increased evaporation and differential temperature effects between wells of the microwell assay plate 10.

Verification of this improvement may be modeled by the Archard Wear Equation, where:
Q=KWL/H; wherein
Q=total volume of debris produced;
K=constant;
W=total normal load;
L=sliding distance; and
H=hardness measurement of the contacting surfaces.

The above equation indicates that wear is linearly proportional to sliding distance. By employing the microplate lid 200 with the bottom frame surface 205B having a substantially flat, substantially planar surface, the measured sliding distance L of the lid 200 over microwell assay plate 10 during shaking operations was found to substantially decrease in comparison to microplate lids featuring a ribbed contact surface, resulting in reduced material wear and/or powder byproduct.

Second, relative to other microplate lid designs, the microplate lid 200 promotes a reduction in air flow across the top of the microwell assay plate 10. Because the bottom frame surface 205B is configured to maintain a substantially continuous contact with the surface of the microwell assay plate 10, the microplate lid 200 acts to seal the wells 11 of the microwell assay plate 10. The substantially continuous contact reduces or eliminates any air passages between the microplate lid 200 and the microwell assay plate 10, thereby reducing or preventing air from flowing from an exterior environment into the space between the microplate lid 200 and the microwell assay plate 10. This reduction/elimination of air flow may reduce the amount of evaporation from the wells 11 of the microwell assay plate 10 and thus increase consistency of assay performance.

FIG. 5A illustrates a side perspective, cross-sectional view of a microwell assay plate 10 and the microplate lid 200 consistent with embodiments hereof. FIG. 5B illustrates a profile of the microplate lid 200 edge having the top surface 204A and the bottom surface 204B as previously described herein. The thickness dimension 217 of the lid frame may be approximately 0.04 in. (or between 0.03 and 0.05 in.). The sidewalls 206 may extend downwards from the top frame surface 205A and bottom frame surface 205B of lid frame 205 at an angle δ between 91-95 degrees with respect to the bottom frame surface 205B, as shown in FIG. 5B. Sidewalls 206 substantially envelop or surround the top edges of the microwell assay plate 10 once the lid 200 is placed over the plate 10. The sidewalls 206 may feature chamfered edges 207 (further illustrated by FIG. 5C), having a bevel angle α of approximately 15 degrees (or between 10 and 20 degrees). The bevel angle α of the chamfered edges 207 is measured with respect to the unchamfered edge of the side wall 206. The bevel angle α may allow for an easier and more accurate fit of the microplate lid 200 over the microwell assay plate 10, as further detailed below. In embodiments, the chamfered edges 207 may be manufactured on the outside surfaces (i.e. facing away from the microwell assay plate 10) of the sidewalls 206, as shown in FIGS. 3C-3D. The chamfered edges 207 may also be manufactured on both the inside and outside surfaces of the sidewall 206.

An advantage of the chamfered edges 207 may additionally facilitate robotic placement operations of the microplate lid 200 over the microwell assay plate 10, as the chamfered edges 207 permit a degree of error forgiveness in placement positioning without consequence of total placement operation failure. A robotic lid placement operation resulting in a slight mis-placement of the microplate lid 200 over the microwell assay plate 10 may result in a correctional “slide” or “shift” of the microplate lid 200 back over the top of microwell assay plate 10, such that the microplate lid 200 and microwell assay plate 10 are substantially aligned. This correctional “slide” may further prevent automated assay system crashes/failures, and/or aborted runs that would otherwise occur from total placement operation failure. The degree of error forgiveness provided by the chamfered edges may further assist in training the automated system to properly grab the microplate lid 200 via an end effector apparatus attached to a robotic arm of the robot, to move the microplate lid 200 towards/over the microwell assay plate 10, and to then properly place the microplate lid 200 over the microwell assay plate 10 so as to completely cover the wells 11. The chamfered edges may also assist in scenarios involving manual handling, where the degree of error forgiveness provided by the chamfered edges may provide an increased ability to handle a higher number of plates at any one time. For example, the chamfered edges 207 of the microplate lid 200 may allow for easier manual stacking and interaction with a higher number of microplate lids, than in scenarios with lids lacking such chamfered edges. In another example, the chamfered edges 207 of the microplate lid 200 may allow for easier, faster, and/or more secure manual positioning of the microplate lid 200 on the microwell assay plate 10 during workflow of a manually conducted assay.

FIG. 6 illustrates a stacking rib feature of the microplate lid 200. A plurality of ribs 203 are disposed at the top surface 201 of the microplate lid 200, specifically on the top frame surface 205A. The plurality of ribs 203 may each include a pair of substantially perpendicular extensions 203A, 203B disposed adjacent to peripheral edges of the top frame surface 205A, for example, at the corners of the top frame surface 205A. The perpendicular extensions 203A, 203B may meet at the corners of the top frame surface 205A, or may be spaced from each other in embodiments. In further embodiments, additional ribs 203 may be provided in any suitable location at the periphery of the top frame surface 205A.

The placement and configuration of the ribs 203 allows several advantages. A few non-limiting examples of these advantages are provided below. As a first example, the ribs 203 facilitate the stacking of multiple microplate lids 200. The ribs 203 may interface with the sidewalls 206 of another microplate lid 200 placed atop the first microplate lid 200, causing alignment between the stacked microplate lids 200 and further creating the spacing height H2 between the top surface 201 and the bottom surface 202 of another lid 200 stacked on top of the first lid, such that the dome does not contact the stacked lid bottom panel surface 204. The ability to stack multiple microplate lids 200 in an easily-storable manner thus results. The arrangement of these ribs 203 further provide the benefit of a more secure stacking arrangement between the microplate lids 200, therefore decreasing the likelihood of tip-over incidents, breakage, or shifting in instances where a plurality of microplate lids 200 are relocated in bulk. Second, secure alignment of the stacked microplate lids 200 facilitates shipping transport as the possibility of the edges of a microplate lid 200 damaging the corners of another microplate lid 200 are reduced due to the more even force distribution permitted by the aligned stacking. Third, the ribs 203 can provide an increased structural integrity of the lid 200, which further bolsters the warp resistant capabilities of the microplate lid 200.

The various features of the microplate lid 200 discussed above may be combined in any combination. In embodiments, the microplate lid 200 may include the dome shape of the lid panel 204 and incorporate the ribs 203 as described herein, and exclude the chamfered edges 207 on the sidewalls. The microplate lid may include the dome shape of the lid panel 204, and exclude the ribs 203 and chamfered edges 207 as described herein. The microplate lid may exclude the dome shape of the lid panel 204, and include the ribs 203 and chamfered edges 207 as described herein. The microplate lid may exclude the dome shape of the lid panel 204 and the ribs 203, and include the chamfered edges 207.

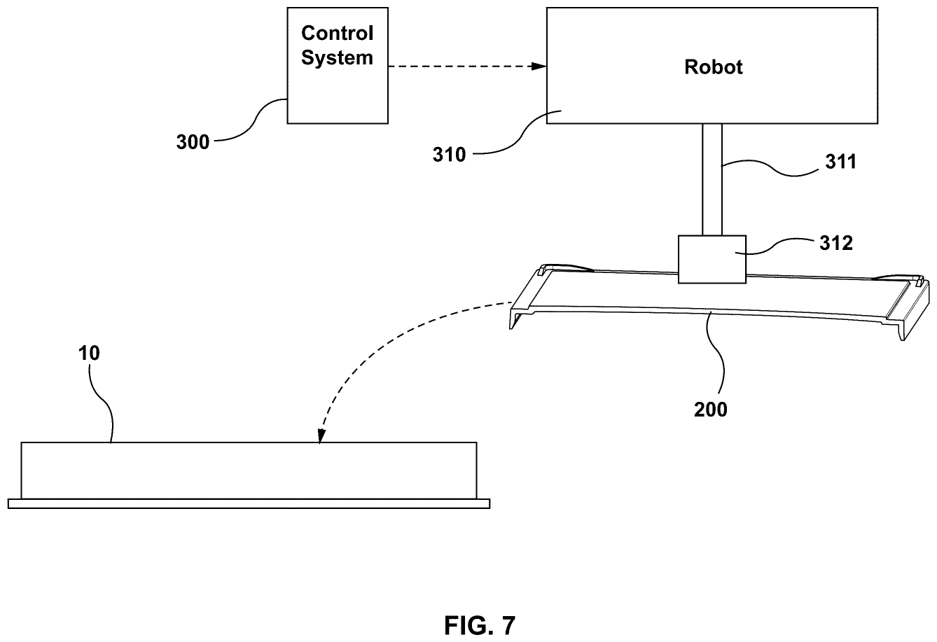

FIG. 7 illustrates an automated assay system for placing microplate lids 200 on microwell assay plates 10. In embodiments, a control system 300 is configured to communicate with a robot 310 having a robot arm 311 that includes or is attached to an end effector apparatus 312. The control system 300 may output a command to the robot 310 to control/move the robot arm 311 to grab microplate lids 200 via the end effector apparatus 312. The end effector apparatus 312 may include, for example, robotic arms or fingers, robotic grippers, such as suction grippers, jaws, claws, lifters, and/or any other suitable end effector. The control system 300 may further command the robot 310 to move the arm 311 and the microplate lid 200 over to the microwell assay plate 10, such that the microplate lid 200 is substantially over the top of the microwell assay plate 10. The control system 300 may further command the robot 310 to then place the microplate lid 200 onto the microwell assay plate 10, such that the microplate lid 200 covers the entirety of the microwell assay plate 10. In embodiments, the microplate lid 200 may be placed partially off-center from the microwell assay plate 10, and subsequently slide into place (i.e. properly fitting over the microwell assay plate 10) via the chamfered edges 207, as previously described in detail above.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described exemplary embodiments. It will also be understood that each feature of each embodiment discussed herein can be used in combination with the features of any other embodiment.

Further embodiments may include:

Embodiment 1. A microplate lid configured to cover a microwell assay plate, comprising: a lid panel having a top panel surface and a bottom panel surface, a lid frame surrounding the lid panel having a top frame surface and a bottom frame surface; and a plurality of sidewalls extending from a periphery of the bottom frame surface; and further comprising at least one feature selected from: a plurality of ribs extending from the top frame surface, chamfered edges disposed at a bottom of the plurality of sidewalls, a convex shape formed by the lid panel, and a substantially continuous contact surface formed by the frame bottom surface.

Embodiment 2. The microplate lid of embodiment 1, wherein the at least one feature includes the convex shape formed by the lid panel, wherein the lid panel is configured to provide a spacing height between 0.03 and 0.07 in. Inches from a center of the microwell assay plate.

Embodiment 3. The microplate lid of embodiment 2, wherein the spacing height is configured to prevent contact between a top surface of the microwell assay plate and the bottom panel surface.

Embodiment 4. The microplate lid of embodiment 2, wherein the lid panel projects from the lid frame according to a curvature.

Embodiment 5. The microplate lid of embodiment 2, wherein the convex shape of the lid panel is formed by a curvature.

Embodiment 6. The microplate lid of embodiment 5, wherein the convex shape of the lid panel is formed by an angle β between the lid frame and the lid panel.

Embodiment 7. The microplate lid of embodiment 2, wherein the lid panel is vertically offset from the lid frame.

Embodiment 8. The microplate lid of embodiment 2, wherein the spacing height of the lid panel is configured to not contact a bottom surface of a stacked lid.

Embodiment 9. The microplate lid of embodiment 1, wherein the at least one feature includes the substantially continuous contact surface formed by the frame bottom surface, and wherein the frame bottom surface is substantially planar.

Embodiment 10. The microplate lid of embodiment 9, wherein the frame bottom surface is configured for contact around substantially all of a perimeter of the microwell assay plate.

Embodiment 11. The microplate lid of embodiment 1, wherein the at least one feature includes the chamfered edges disposed at the bottom of the plurality of sidewalls, and wherein the plurality of sidewalls are arranged at an angle between 91 and 95 degrees with respect to the frame bottom surface.

Embodiment 12. The microplate lid of embodiment 11, wherein the plurality of sidewalls provide a continuous circumference.

Embodiment 13. The microplate lid of embodiment 11, wherein the chamfered edges have a bevel angle between 10 and 20 degrees.

Embodiment 14. The microplate lid of embodiment 11, wherein the chamfered edges are configured to facilitate robotic positioning of the microplate lid.

Embodiment 15. The microplate lid of embodiment 1, wherein the at least one feature includes the plurality of ribs, and wherein the plurality of ribs are configured to receive a stacked second microplate lid.

Embodiment 16. The microplate lid of embodiment 15, wherein each of the plurality of ribs includes a pair of perpendicular extensions.

Embodiment 17. The microplate lid of embodiment 15, wherein the stacked second microplate lid stacked on top of the microplate lid has a second spacing height between the top surface of the microplate lid and a bottom surface of the stacked second microplate lid, such that the convex shape of the lid panel of the microplate lid does not contact the stacked second microplate lid bottom surface.

Embodiment 18. An assay system comprising: a microwell assay plate including a plurality of sample wells; and a microplate lid configured to cover the microwell assay plate, the microplate lid comprising: a lid panel having a top panel surface and a bottom panel surface, a lid frame surrounding the lid panel having a top frame surface and a bottom frame surface; and a plurality of sidewalls extending from a periphery of the bottom frame surface; and further comprising at least one feature selected from: a plurality of ribs extending from the top frame surface, chamfered edges disposed at a bottom of the plurality of sidewalls, a convex shape formed by the lid panel, and a substantially continuous contact surface formed by the frame bottom surface.

Embodiment 19. The assay system of embodiment 18, wherein the at least one feature includes the convex shape formed by the lid panel, wherein the lid panel is configured to provide a spacing height between 0.03 and 0.07 in. Inches from a center of the microwell assay plate.

Embodiment 20. The assay system of embodiment 19, wherein the spacing height is configured to prevent contact between a top surface of the microwell assay plate and the bottom panel surface.

Embodiment 21. The assay system of embodiment 19, wherein the lid panel projects from the lid frame according to a curvature.

Embodiment 22. The assay system of embodiment 19, wherein the convex shape of the lid panel is formed by a curvature.

Embodiment 23. The assay system of embodiment 22, wherein the convex shape of the lid panel is formed by an angle β between the lid frame and the lid panel.

Embodiment 24. The assay system of embodiment 19, wherein the lid panel is vertically offset from the lid frame.

Embodiment 25. The assay system of embodiment 19, wherein the spacing height of the lid panel is configured to not contact a bottom surface of a stacked lid.

Embodiment 26. The assay system of embodiment 18, wherein the at least one feature includes the substantially continuous contact surface formed by the frame bottom surface, and wherein the frame bottom surface is substantially planar.

Embodiment 27. The assay system of embodiment 26, wherein the frame bottom surface is configured for contact around substantially all of a perimeter of the microwell assay plate.

Embodiment 28. The assay system of embodiment 18, wherein the at least one feature includes the chamfered edges disposed at the bottom of the plurality of sidewalls, and wherein the plurality of sidewalls are arranged at an angle between 91 and 95 degrees with respect to the frame bottom surface.

Embodiment 29. The assay system of embodiment 28, wherein the plurality of sidewalls provide a continuous circumference.

Embodiment 30. The assay system of embodiment 28, wherein the chamfered edges have a bevel angle between 10 and 20 degrees.

Embodiment 31. The assay system of embodiment 28, wherein the chamfered edges are configured to facilitate robotic positioning of the microplate lid.

Embodiment 32. The assay system of embodiment 18, wherein the at least one feature includes the plurality of ribs, and wherein the plurality of ribs are configured to receive a stacked second microplate lid.

Embodiment 33. The assay system of embodiment 32, wherein each of the plurality of ribs includes a pair of perpendicular extensions.

Embodiment 34. The assay system of embodiment 32, wherein the stacked second microplate lid stacked on top of the microplate lid has a second spacing height between the top surface of the microplate lid and a bottom surface of the stacked second microplate lid, such that the convex shape of the lid panel of the microplate lid does not contact the stacked second microplate lid bottom surface.

Embodiment 35. An automated assay system, comprising: a control system configured to communicate with a robot having a robot arm that includes or is attached to an end effector apparatus; a microwell assay plate including a plurality of wells for receiving samples therein; a microplate lid configured to cover the microwell assay plate, the microplate lid comprising: a lid panel having a top panel surface and a bottom panel surface, a lid frame surrounding the lid panel having a top frame surface and a bottom frame surface, and a plurality of sidewalls extending from a periphery of the bottom frame surface, the plurality of sidewalls further comprising at least one feature selected from: a plurality of ribs extending from the top frame surface, chamfered edges disposed at a bottom of the plurality of sidewalls, a convex shape formed by the lid panel, and a substantially continuous contact surface formed by the frame bottom surface; wherein the control system outputs a command controlling the robot arm to grab the microplate lid via the end effector apparatus, move the microplate lid over to a microwell assay plate, and place the microplate lid over the microwell assay plate.

1. A microplate lid configured to cover a microwell assay plate, comprising:
a lid panel having a top panel surface and a bottom panel surface,
a lid frame surrounding the lid panel having a top frame surface and a bottom frame surface; and
a plurality of sidewalls extending from a periphery of the bottom frame surface; and
further comprising at least one feature selected from:
a plurality of ribs extending from the top frame surface,
chamfered edges disposed at a bottom of the plurality of sidewalls,
a convex shape formed by the lid panel, and
a substantially continuous contact surface formed by the bottom frame surface.

2. The microplate lid of claim 1, wherein the at least one feature includes the convex shape formed by the lid panel, wherein the lid panel is configured to provide a spacing height between 0.03 and 0.07 in. inches from a center of the microwell assay plate.

3. The microplate lid of claim 2, wherein the spacing height is configured to prevent contact between a top surface of the microwell assay plate and the bottom panel surface.

4. The microplate lid of claim 2, wherein the lid panel projects from the lid frame according to a curvature.

5. The microplate lid of claim 2, wherein the convex shape of the lid panel is formed by a curvature.

6. The microplate lid of claim 5, wherein the convex shape of the lid panel is formed by an angle β between the lid frame and the lid panel.

7. The microplate lid of claim 2, wherein the lid panel is vertically offset from the lid frame.

8. The microplate lid of claim 2, wherein the spacing height of the lid panel is configured to not contact a bottom surface of a stacked lid.

9. The microplate lid of claim 1, wherein the at least one feature includes the substantially continuous contact surface formed by the bottom frame surface, and
wherein the bottom frame surface is substantially planar.

10. The microplate lid of claim 9, wherein the bottom frame surface is configured for contact around substantially all of a perimeter of the microwell assay plate.

11. The microplate lid of claim 1, wherein the at least one feature includes the chamfered edges disposed at the bottom of the plurality of sidewalls, and wherein the plurality of sidewalls are arranged at an angle between 91 and 95 degrees with respect to the bottom frame surface.

12. The microplate lid of claim 11, wherein the plurality of sidewalls provide a continuous circumference.

13. The microplate lid of claim 11, wherein the chamfered edges have a bevel angle between 10 and 20 degrees.

14. The microplate lid of claim 11, wherein the chamfered edges are configured to facilitate robotic positioning of the microplate lid.

15. The microplate lid of claim 1, wherein the at least one feature includes the plurality of ribs, and wherein the plurality of ribs are configured to receive a stacked second microplate lid.

16. The microplate lid of claim 15, wherein each of the plurality of ribs includes a pair of perpendicular extensions.

17. The microplate lid of claim 15, wherein the stacked second microplate lid stacked on top of the microplate lid has a second spacing height between the top panel surface of the microplate lid and a bottom surface of the stacked second microplate lid, such that the convex shape of the lid panel of the microplate lid does not contact the stacked second microplate lid bottom surface.

18. An assay system comprising:
a microwell assay plate including a plurality of sample wells; and
a microplate lid configured to cover the microwell assay plate, the microplate lid comprising:
a lid panel having a top panel surface and a bottom panel surface, a lid frame surrounding the lid panel having a top frame surface and a bottom frame surface; and
a plurality of sidewalls extending from a periphery of the bottom frame surface; and
further comprising at least one feature selected from:
a plurality of ribs extending from the top frame surface,
chamfered edges disposed at a bottom of the plurality of sidewalls,
a convex shape formed by the lid panel, and
a substantially continuous contact surface formed by the bottom frame surface.

19. The assay system of claim 18, wherein the at least one feature includes the convex shape formed by the lid panel, wherein the lid panel is configured to provide a spacing height between 0.03 and 0.07 in. inches from a center of the microwell assay plate.

20.-34. (canceled)

35. An automated assay system, comprising:
a control system configured to communicate with a robot having a robot arm that includes or is attached to an end effector apparatus;
a microwell assay plate including a plurality of wells for receiving samples therein;
a microplate lid configured to cover the microwell assay plate, the microplate lid comprising:
a lid panel having a top panel surface and a bottom panel surface,
a lid frame surrounding the lid panel having a top frame surface and a bottom frame surface, and
a plurality of sidewalls extending from a periphery of the bottom frame surface, the plurality of sidewalls further comprising at least one feature selected from:
a plurality of ribs extending from the top frame surface,
chamfered edges disposed at a bottom of the plurality of sidewalls,
a convex shape formed by the lid panel, and
a substantially continuous contact surface formed by the bottom frame surface;
wherein the control system outputs a command controlling the robot arm to grab the microplate lid via the end effector apparatus, move the microplate lid over to the microwell assay plate, and place the microplate lid over the microwell assay plate.
